(19) United States
(12) Patent Application Publication
(10) Pub. No.: US 2006/0030740 A1
(43) Pub. Date: Feb. 9, 2006

(54) Process for preparing polyalkylene glycol diethers

(75) Inventors: Achim Stankowiak, Altoetting (DE); Alexander Snell, Altoetting (DE)

Correspondence Address: CLARIANT CORPORATION;INTELLECTUAL PROPERTY DEPARTMENT, 4000 MONROE ROAD, CHARLOTTE, NC, 28205, US

(73) Assignee: Clariant GmbH

(21) Appl. No.: 11/197,984

(22) Filed: Aug. 5, 2005

(30) Foreign Application Priority Data
Aug. 6, 2004 (DE) 102004038157.7

(51) Int. Cl. C07C 43/11

(52) U.S. Cl. 568/618

(57) ABSTRACT

The invention relates to a process for preparing alkylene glycol diethers of the formula (I) 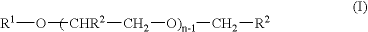
by reacting compounds of the formula (II) 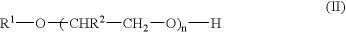
in which R¹ is hydrogen or C₁ to C₃ alkyl, R² is hydrogen, CH₃ or CH₂—CH₃ and n is from 5 to 500, in the liquid phase in the presence of Raney nickel at temperatures between 170 and 300° C.

The present invention relates to a process for preparing catenated alkylene glycol diethers having a molecular weight of at least 250 g/mol.

Alkylene glycol diethers have been used for a long time as polar, aprotic, inert solvents. High molecular weight alkylene glycol diethers find use in particular in electrochemistry, as high-boiling solvents and as linear crown ethers in phase-transfer catalysis.

For their preparation, what are known as indirect processes, for example the Williamson ether synthesis (K. Weissermel, H. J. Arpe, “Industrielle Organische Chemie” [Industrial organic chemistry], 1998, page 179) or the hydrogenation of diglycol ether formal (DE-A 24 34 057) are industrially employed or described. However, both processes have disadvantages: the two-stage Williamson ether synthesis has low economic viability by virtue of the stoichiometric consumption of chlorine and alkali, and also the removal of the water of reaction and sodium chloride which forms. The hydrogenation of formal is carried out under high pressure, which has the prerequisite of high capital costs in the plant construction and is therefore unsuitable for small production amounts.

In what are known as direct processes, alkylene oxide is inserted into a catenated ether in the presence of Lewis acids such as BF₃ (U.S. Pat. No. 4,146,736 and DE-A 26 40 505 in conjunction with DE-A 31 28 962) or SnCl₄ (DE-A 30 25 434). The disadvantage of these processes is that large amounts of cyclic by-products, for example dioxane or dioxolane, are unavoidably formed. Furthermore, these processes cannot be applied to relatively long-chain polyalkylene glycol ethers (high proportion of by-products).

An alternative synthetic means is the catalytic deformylation of glycols and methyl glycols:

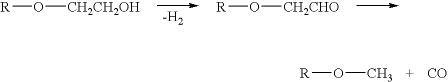

The patent DE 2 900 279 describes this synthetic route for the first time by the reaction of polyethylene glycols or polyethylene glycol monomethyl ethers in the gas phase at 250-500° C. in the presence of supported palladium, platinum, rhodium, ruthenium or iridium catalysts and hydrogen. A Japanese patent JP 60028429 describes the reaction of C₄ and longer-chain monoalkyl ethers using a nickel/rhenium catalyst supported on γ-alumina. In this process too, hydrogen is supplied continuously. Likewise known is the hydrogenation of secondary hydroxyl groups with hydrogen at standard pressure using supported nickel catalysts (DE-A 38 02 783). In this process, the synthesis explicitly does not succeed when Raney nickel is used.

The patent U.S. Pat. No. 3,428,692 discloses that it is possible by heating C₆- to C₁₂-chain monoalkyl and monophenyl ethers to 200-300° C. in the presence of nickel and cobalt catalysts to prepare the corresponding deformylated methyl-capped ethoxylates. However, this forms mixtures of the desired methyl ethers with ethoxylates which have not been fully converted and 20-30% of undefined aldehyde compounds. EP 0 043420 describes a similar process using palladium, platinum or rhodium catalysts supported on Al₂O or SiO₂.

All processes described in the current prior art either have low selectivity or else are technically very complex and therefore uneconomic for the preparation of relatively long-chain alkylene glycol diethers. The object arising therefrom is achieved in accordance with the invention according to the specifications of the claims.

Surprisingly, it is possible to convert relatively long-chain alkylene glycols and alkylene glycol monoethers to the desired alkylene glycol diethers in a simple slurry process by nickel catalysis. The synthesis succeeds quantitatively (>99%) and without formation of by-products. After the reaction, the catalyst can be removed fully in a simple filtration step (<1 ppm of metal).

The invention thus provides a process for preparing alkylene glycol diethers of the formula (I) 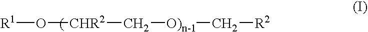
by reacting compounds of the formula (II) 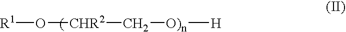
in which R¹ is hydrogen or C₁ to C₃ alkyl, R² is hydrogen, CH₃ or CH₂—CH₃ and n is from 5 to 500, in the liquid phase in the presence of Raney nickel at temperatures between 170 and 300° C.

Suitable catalysts are pure Raney nickel catalysts and mixtures of Raney nickel with palladium, platinum or rhodium catalysts. Preference is given to using pure Raney nickel. The conversion over the catalysts is effected preferably at from 200 to 250° C. The reaction is generally carried out at standard pressure, but it is also possible to work in elevated or reduced pressure. The reaction time is generally between 4 and 10 hours.

R¹ is preferably H or methyl.

R² is preferably hydrogen.

n is preferably from 15 to 300.

The Raney nickel catalyst may contain up to 10% by weight of other metals, for example palladium, copper, chromium, cobalt, platinum, rhodium, ruthenium or iridium.

The process according to the invention will now be illustrated in detail with reference to some examples:

### Example

### Synthesis of Polyglycol Dimethyl Ether Having a Molar Mass of Approx. 500

In a 250 ml three-neck flask, 361.7 g of polyglycol monomethyl ether (molar mass approx. 500 g/mol), 12.3 g of palladium on activated carbon and 19.4 g of anhydrous Raney nickel are stirred vigorously at 230° C. under protective gas. After 8 hours of reaction time, the reaction mixture is filtered through silica gel at 80° C. The conversion is 98.6%. In the product, no nickel can be detected by means of atomic absorption spectroscopy (AAS).

### Example 2

### Synthesis of Polyglycol Dimethyl Ether Having a Molar Mass of Approx. 2000

In a 250 ml three-neck flask, 399.5 g of polyglycol monomethyl ether (molar mass approx. 2000 g/mol) and 31.0 g of anhydrous Raney nickel are stirred vigorously at 230° C. under protective gas. After 6 hours of reaction time, the reaction mixture is filtered through silica gel at 80° C. The conversion is 99.3%. In the product, no nickel can be detected by means of atomic absorption spectroscopy (AAS).

### Example 3

### Synthesis of Polyglycol Dimethyl Ether Having a Molar Mass of Approx. 4000

In a 250 ml three-neck flask, 395.5 g of polyglycol monomethyl ether (molar mass approx. 4000 g/mol) and 30.6 g of anhydrous Raney nickel are stirred vigorously at 230° C. under protective gas. After 8 hours of reaction time, the reaction mixture is filtered through silica gel at 80° C. The conversion is 98.8%.

### Example 4

### Synthesis of Polyglycol Dimethyl Ether Having a Molar Mass of Approx. 10 000

In a 250 ml three-neck flask, 331.5 g of polyglycol monomethyl ether (molar mass approx. 10 000 g/mol) and 18.7 g of anhydrous Raney nickel are stirred vigorously at 200° C. under protective gas. After 8 hours of reaction time, the reaction mixture is filtered through silica gel at 80° C. The conversion is 91.2%.

### Example 5

### Synthesis of Polyglycol Dimethyl Ether Having a Molar Mass of Approx. 10 000

In a 250 ml three-neck flask, 332.4 g of polyglycol monomethyl ether (molar mass approx. 10 000 g/mol) and 18.3 g of anhydrous Raney nickel are stirred vigorously at 230° C. under protective gas. After 8 hours of reaction time, the reaction mixture is filtered through silica gel. The conversion is 99.0%.

1. A process for preparing alkylene glycol diethers of the formula (I) 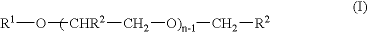
by reacting compounds of the formula (II) 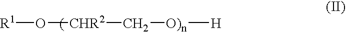
in which R¹ is hydrogen or C₁ to C₃ alkyl, R² is hydrogen, CH₃ or CH₂—CH₃ and n is from 5 to 500, in the liquid phase in the presence of Raney nickel at temperatures between 170 and 300° C.

2. The process as claimed in claim 1, in which R¹ is H or methyl.

3. The process as claimed in claim 1, in which R¹ is methyl.

4. The process of claim 1, in which R² is H.

5. The process of claim 1, in which n is from 15 to 300.

6. The process of claim 1, in which the Raney nickel catalyst further comprises up to 10% by weight of a metal selected from the group consisting of palladium, copper, chromium, cobalt, platinum, rhodium, iridium, and mixtures thereof.
